(19) United States
(12) Patent Application Publication
(10) Pub. No.: US 2008/0077223 A1
(43) Pub. Date: Mar. 27, 2008

(54) Stent delivery system with improved deliverabilty features

(76) Inventors: Robert E. Fischell, Dayton, MD (US); Fernando DiCaprio, St. Paul, MN (US); Tim A. Fischell, Kalamazoo, MI (US); David R. Fischell, Fair Haven, NJ (US); Scott J.S. Fischell, Glenelg, MD (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/524,460

(22) Filed: Sep. 21, 2006

(51) Int. Cl. A61F 2/06

(52) U.S. Cl. 623/1.11

(57) ABSTRACT

A stent delivery system for placing a stent within a vessel of a human body. The stent delivery system includes an elongated tube that extends for most of the length of the stent delivery system and is attached to a distal end of a proximal section of an inflatable balloon. A stent is then co-axially mounted on the inflatable balloon. A front section of the stent delivery system includes a small angle cone having a lubricious outer surface. The front section is then attached to a cylindrical distal portion of the balloon and has an outside diameter at the proximal end of the small angled cone which is approximately the same diameter as the outer diameter of the stent. A core wire extends within the inflatable balloon and further into the front section of the stent delivery system.

### FIELD OF USE

This invention is in the field of methods and devices for placing a stent within a vessel of a human body.

### BACKGROUND OF THE INVENTION

Stents are well known devices for placement in vessels of the human body to obtain and maintain patency of that vessel. The greatest use for stents has been for placement within a stenosis in a coronary artery. When a stent is used for treating a coronary artery stenosis, it has always been necessary to first place a guidewire through the stenosis. The next step in the stenting procedure may be to pre-dilate the stenosis with a balloon angioplasty catheter that is advanced over that guidewire. The catheter may be of the over-the-wire or rapid exchange variety. The balloon angioplasty catheter is then removed and a stent delivery system which includes the stent is advanced over the guidewire, and the stent is then deployed at the site of the dilated stenosis.

Recent improvements in the design of stent delivery systems have made it possible to eliminate the step of pre-dilatation for the treatment of many classes of stenoses. The delivery of a stent to the site of a stenosis without pre-dilatation has been commonly referred to as “direct stenting.” However, even with direct stenting, a guidewire is still required as a precursor to advancing the stent delivery system over that guidewire to place the stent at the site of a stenosis. Placing the guidewire requires additional cost and additional procedure time. Furthermore, most coronary stenoses are sufficiently tight so that direct stenting is accomplished in only approximately 20% of all stenting procedures.

U.S. Pat. No. 6,375,660 by Fischell et al describes a means to decrease the outside diameter of a stent to be delivered into a stenosis by eliminating the need for a guide wire to pass through an inner tube both of which lie within the balloon used for delivering the stent. Such a stent delivery system can reduce the outside diameter (sometimes called the profile) of the stent by as much as 25%. This provides a dramatic improvement in delivering the stent into a tight stenosis.

In U.S. Pat. No. 6,936,065, Khan et al describe an improved stent delivery system that has a significantly reduced outer diameter for the stent on an inflatable balloon. This design has certain advantages as compared to the conventional stent delivery system that has an inner tube and a guide wire that lie within the balloon. However, the Khan et al design does not have a distal portion which minimizes the frictional forces as it is pushed through a tight stenosis. Furthermore, the Khan et al invention uses a wound wire over the core wire at the front section of the stent delivery system. Although this is typical for any guide wire, this wound wire can become damaged when placed through tortuous coronary vasculature, and when damaged, it becomes irreparable. Still further, the Khan et al design does not have a built-in valve connection at the stent delivery system's proximal portion that minimizes the time and cost for performing a stenting procedure.

### SUMMARY OF THE INVENTION

Disclosed herein is a stent-on-a-wire stent delivery system that can provide significant improvement in deliverability of a stent into a very tight stenosis. Because the system described herein eliminates the need for a guide wire and also eliminates the need for pre-dilitation of any stenosis, the use of this system results in a considerable saving in cost and the time to perform the procedure of stenting an arterial (or any) stenosis. Although this disclosure will emphasize the use of this system in coronary arteries, it should be understood that this system can be used for the dilitation of any vessel of the human body in which patency can be restored by means of stenting. These vessels include, but are not limited to, peripheral arteries (particularly below the knee), renal arteries, arteries in the brain and other vessels of the human body such as fallopian tubes, the ureter, the urethra, etc.

An important feature of the present invention is that the front section of the stent-on-a-wire system is not a guide wire that is covered with a wound wire, but is in fact a shape memory alloy core wire that is covered with a highly lubricious polymer coating. The most distal portion of this front section has an essentially uniform diameter that joins continuously with a proximal portion of the front section that is a cone with an extraordinarily small cone angle. The entire outer surface of the polymer covering of the front section of the stent-on-a-wire system is a polymer material that has an extremely lubricious outer surface so that it can be readily pushed into a very tight stenosis. By eliminating a wire wound around a core wire as is typical for a guide wire and is shown as the front section of the Khan et al patent, the polymer coating around the core wire of the present invention does not have the failure mode of the wound wire coming off the core wire. This design improves both the utility and the reliability of the stent delivery system of the present invention.

The proximal end of the conical proximal portion of the front section of the stent delivery system would have an outside diameter that is approximately equal to the outside diameter of the stent mounted on the balloon of the stent-on-a-wire stent delivery system. Between the proximal end of the cone and the distal end of the stent would be a distal elastomer band that is placed over that distal portion of the balloon that assumes a conical shape when the balloon is inflated. The outside diameter of this is equal to the outside diameter of the proximal end of the cone that covers the front section of the core wire and is also equal to the outside diameter of the stent that is mounted onto the balloon of the stent delivery system. Thus there is a smooth transition of uniform outside diameter from the proximal end of the small angle cone of the front section of the stent delivery system and the distal end of the stent mounted on the balloon. Thus there is no protrusion at the stent's proximal end that could act as an impediment to a smooth passage through a tight stenosis. The outer surface of the elastomer band would be lubricity coated to further improve the ability of the stent delivery system to pass through a tight stenosis.

Just proximal to the proximal end of the stent a proximal elastomer band could be placed over the balloon to make a smooth transition from the outside diameter of the proximal end of the stent to the distal end of the tube onto which is mounted a cylindrical proximal portion of the balloon. Both the distal and proximal elastomer bands would optimally have a lubricious coating that decreases friction as the stent delivery system is pushed through a tight stenosis.

The core wire of the stent-on-a-wire stent delivery system extends from the distal tip of the stent delivery system through to the distal end of a handle that forms a proximal portion of the stent delivery system. As described in the Khan et al patent referenced above, the core wire has its smallest diameter at the distal portion of the core wire and has an increased diameter for most of the proximal length of the core wire. Thus the pushability of the stent delivery system is enhanced by the core wire that extends for essentially the entire length of the stent delivery system. However, as taught in the Fischell et al U.S. Pat. No. 6,375,660, it should be understood that the core wire could extend only from the distal end of the stent delivery system to the distal end of the cylindrical tube that extends for most of the length of the stent delivery system.

The front section of the core wire is preferably fabricated from a shape memory alloy such as Nitinol with a transition temperature that is clearly above body temperature. Specifically, for Nitinol, the front section of the core wire should have an austenitic transition start temperature above body temperature so that the distal core wire will remain malleable at body temperature and the austenitic finish temperature such that the distal core wire will be heat memory shape recoverable at a temperature well above body temperature (e.g. 120° F.). Thus, the interventional cardiologist can form a shape for the stent delivery system's distal front section that will assist in delivering the stent though a tortuous artery. Because the austenitic transition start temperature is above body temperature, any bend in the core wire that is at the center of the front section of the stent delivery system will maintain that bend within the body. If the front section of the stent delivery system becomes inadvertently distorted as the stent delivery system is pushed though some coronary vasculature, the system can be removed from the body and the front section can then be heated to above the austenitic transition finish temperature so that the Nitinol core wire will return to its pre-set memory shape. When that front section is then cooled below the austenitic transition start temperature, it again would be able to be shaped by the interventional cardiologist as required to continue a placement of the stent. It is envisioned that the memory shape of the distal core wire may be either straight or include one or more bends which would assist in device delivery and be heat recoverable.

The entire length of the core wire that lies proximal to the distal end of the balloon would have an austenitic transition finish temperature that lies below body temperature. A typical austenitic finish temperature for the length of the core wire that lies proximal to the balloon's distal end would be between 70° F. to 90° F. Thus, all the length of the core wire except that portion that is in the front section of the stent delivery system would always automatically straighten itself out because inside the body, the core wire would be at body temperature. It is also envisioned that having the proximal core wire have its austenitic transition finish temperature below room temperature would cause the entire length of the proximal wire including the portion that lies outside of the body to be in a superelastic state which would improve pushability. In this case, the austenitic finish temperature would be below 60° F. It is also envisioned that the distal half of the proximal core wire could have a higher austenitic finish temperature than the proximal half which will extend outside of the patient's body.

Another novel feature of the stent delivery system is the handle at the proximal portion of the stent delivery system. This handle is used by the interventional cardiologist to steer the front section of the stent delivery system through even the tortuous arterial vasculature of a human heart. This handle includes a built-in valve that allows the interventional cardiologist to perform a stenting procedure without the use of a stop cock attached to the proximal end of the handle. This design saves the time it takes to take a stop cock out of its package and attach it to the handle and also saves the cost of the stop cock.

Thus one object of the present invention is to have a stent delivery system that can penetrate an extremely tight stenosis and deliver a stent into that stenosis without the need for pre-dilation with an angioplasty balloon.

Another object of this invention is to utilize a front section of the stent delivery system that has a lubricous polymer cone that has an outside diameter at its proximal end that is approximately equal to the outside diameter of the stent mounted onto the balloon thus improving the capability of the stent delivery system to penetrate a tight stenosis.

Still another object of this invention is to have a core wire that extends for most of the length of the stent delivery system with the transition temperature being above body temperature at the front section of the core wire and below body temperature for all parts of the core wire that lie proximal to the distal end of the balloon.

Still another object of this invention is to have a distal elastomer band that has a lubricious outer surface and lies between the proximal end of the polymer cone of the front section and the distal end of the stent.

Still another object of this invention is to have a valve formed into the handle at the proximal portion of the stent delivery system that eliminates the need for a separate stopcock that would otherwise have to be attached to the proximal end of the handle.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a longitudinal cross section of a distal portion of the stent delivery system.

FIG. 1B is an enlarged view of a distal portion of the stent delivery system.

FIG. 2 is a longitudinal cross section of a proximal portion of the stent delivery system.

FIG. 3 is a transverse cross section of the handle of the stent delivery system at section 3-3 of FIG. 2.

### DETAILED DESCRIPTION OF THE INVENTION

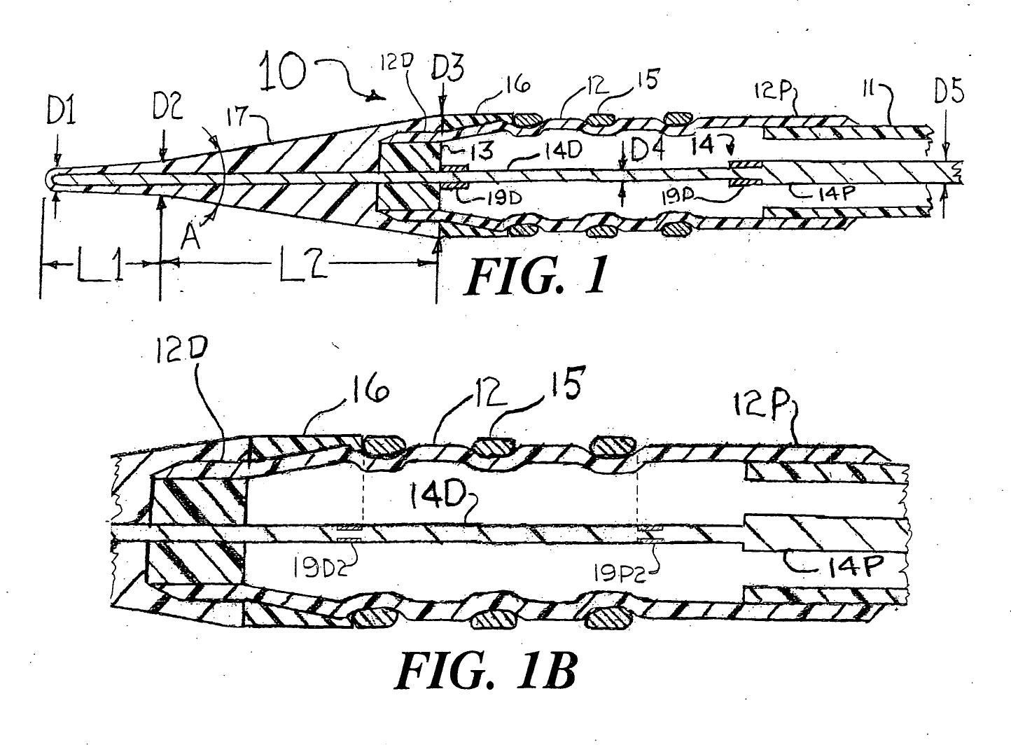

FIG. 1 is a longitudinal cross section of a distal portion of a stent delivery system 10 that has an cylindrical tube 11, a balloon 12, a balloon connector 13, a core wire 14, a stent 15, an elastomer band 16, and a front section having a small angle cone 17, a distal radiopaque marker band 19D and a proximal radiopaque marker band 19P. The cylindrical tube 11 would be formed from a thin-walled polymer that could include metal reinforcement (such as a mesh, braid, strands, etc . . . ) or, at least some of its length could be a thin-walled hypo tube or a hollow strand configuration. The balloon 12 would be a typical balloon as is well known in the art for deploying the stent 15 into an arterial stenosis. Only the outer layer of the folded balloon 12 is shown in FIG. 1. For the sake of clearly visualizing the concepts of the present invention, the scaling of FIG. 1 is such that the radial dimensions of the stent delivery system 10 are very much enlarged as compared to the scaling of the longitudinal dimensions.

A novel feature of the stent delivery system 10 is the front section that has an inner core wire 14D which is a shape memory alloy such as Nitinol that has a transition temperature higher than body temperature. Specifically, for Nitinol, the front section of the core wire should have an austenitic transition start temperature above body temperature so that the distal core wire will remain malleable at body temperature and an austenitic transition finish temperature such that the distal core wire will be heat memory shape recoverable at a temperature well above body temperature (e.g., 120° F.). Thus, any curvature that the interventional cardiologist places into the front section prior to placement into a human body will be retained. If the front section becomes inadvertently bent after placement in the human body, then exposure to a temperature of about 120° F. or higher will return the front section to its pre-set memory shape. When the front section then cools below body temperature, the front section can be reshaped by the interventional cardiologist and advanced again through the human body. The distal wire section 14D of the core wire 14 that lies within the front section of the stent delivery system 10 will, as described above, have a transition temperature that is higher than body temperature. However, that portion of the core wire section 14D that lies within the balloon 12 will have a transition temperature that is below body temperature. Thus, the portion of the distal wire section 14D within the balloon 12 will automatically remain straight as it is pushed through the human body because it will be at body temperature. The section of the core wire 14D that lies within the balloon connector 13 can be that portion of the core wire where the transition temperature changes from higher than body temperature for the core wire 14D distal to the distal end of the balloon connector 13 and lower than body temperature for that portion of the core wire 14D that lies proximal to the proximal end of the balloon connector 13.

The core wire 14 has a proximal wire section 14P that extends for most of the length of the stent delivery system 10. This entire proximal wire section 14P has a transition temperature that is just below body temperature so that it always remains straight as it is pushed through the human body. The diameter D4 of the distal wire section 14D should be approximately 0.007±0.003 inches. This smaller diameter D4 is necessary so that the balloon 12 can be tightly wrapped around the distal wire section 14D and the outside diameter of the balloon 12 will be smaller than if the distal wire section 14D was (let us say) 0.014 inches which is the typical diameter of a coronary guide wire. A smaller outside diameter for the balloon 12 allows for a smaller outside diameter for the stent 15. This smaller outside diameter for the stent 15 (that is, a lower profile) is one of the most important objectives of this invention.

The proximal core wire section 14P would typically have an outside diameter D5 that is typically 0.014±0.005 inches. This increased diameter provides additional pushability for the stent delivery system 10. Just distal to the distal end of the proximal wire section 14P is the proximal radiopaque marker band 19P and just proximal to the balloon connector 13 lies the distal radiopaque marker band 19D. The stent 15 is centered between these two marker bands so the interventional cardiologist can accurately place the stent 15 across an arterial stenosis. It should be noted that the marker bands do not lie directly under the stent 15 which would be a more conventional design The reason for this is that the outside diameter of the marker bands 19P and 19D would be large enough so as to increase the profile of the stent 15. Maintaining the lowest possible profile for the stent 15 is one of the most important objectives of this invention.

Although the core wire 14 is shown to have a distal wire section 14D and a proximal wire section 14P each with a different diameter, it should be understood that the core wire 14 could have three or more different diameters along its length. Also it is envisioned that different sections of the core wire 14 could be made from a tube into which other sections of the core wire could be inserted and fixedly attached. This tube could be very short and made from (for example) steel or Nitinol. Also, it may be less expensive to make the proximal wire section 14P from stainless steel or another material and that would still be an adequate structure to provide good pushability for the stent delivery system 10. Still further, it should be understood that the entire core wire 14 could be made in one or more sections from a metal that does not have a shape memory characteristic.

The balloon 12 is fixedly attached at its cylindrical proximal portion 12P to the cylindrical tube 11 and is joined at its cylindrical distal portion 12D to the balloon connector 13. The balloon connector 13 would typically be made from a polymer cylinder that is joined with a shrink fit or thermally or adhesively bonded to the distal wire section 14D. The outside diameter of the balloon connector 13 would typically be approximately the same diameter as the outside diameter of the cylindrical tube 11. One goal of the design of the stent delivery system 10 is to have an outside diameter of the stent 15 that is equal to or smaller than 0.030 inches. Therefore, the outside diameter of the proximal balloon connector 13 and the cylindrical tube 11 should be less than approximately 0.026 inches.

The stent 15 could be made from stainless steel or from a cobalt-chromium alloy such as L605 or from tantalum or from any other metal that has reasonably high radiopacity. It is also possible to place a bioabsorbable stent 15 onto the balloon 12. If the alloy L605 is used, then optimally it should be heat treated to have a smaller grain size. Also, it is envisioned that the stent 15 would elute a drug that prevents restenosis such as Taxol, sirolimus or a sirolimus analog. Still further, an antithrombogenic coating for the stent 15, using for example heparin or phosphorylcholine, would be useful to prevent acute and subacute thrombosis. A combination of an antithrombogenic coating and elution of an anti-restenosis drug is also envisioned.

A very important novel feature of this invention is the design of the front section of the stent delivery system 10. Specifically, the distal wire section 14D is at the center of the front section surrounded by a polymer material that is designed to be, or by its coating to be extraordinarily lubricious. For example, the polymer of the cone 17 could be made from polyurethane, polyethylene, Nylon, PTFE, polypropylene, PeBax, etc. Furthermore, as is well known in the art of lubricious polymer surfaces, whatever polymer material is used for the cone 17 could have a hydrophilic coating applied such as polyvinyl-pyrrolidone (PVP). Such a hydrophilic coating would aid significantly in improving the lubricity of the cone 17 and/or the elastomer band 16. Ideally the coefficient of friction for the small angle cone 17 becomes even lower in the presence of blood.

The front portion of the front section having a length L1 can be of uniform diameter or it can have a tapered shape with a distal diameter D1 of approximately 0.012±0.002 inches and a proximal diameter D2 of approximately 0.014±0.002 inches. The distal diameter of the small angle cone 17 is the same D2 but the proximal diameter of the small angle cone 17, D3, should be approximately equal to the outside diameter of the stent 15. For an outside diameter of 0.030 inches for the stent 15, the diameter D3 would be equal to 0.030 inches. If the outside diameter of the stent 15 would be less than 0.030 inches, then the diameter D3 would also be less than 0.030. In any case, the diameter D3 for the cone 17 would be essentially equal to the outside diameter of whatever stent 15 is placed on the balloon 12.

If the diameter D2 is its nominal value of 0.014 inches and the diameter D3 is 0.030 inches, then the apex angle “A” of the small angle cone 17 would be 1.55 degrees which is indeed a very small angle. The combination of a very small apex angle “A” and a very lubricious surface for the cone 17 allows the stent delivery system 10 to be readily pushable through even a very tight stenosis. The apex angle “A” should certainly be less than 10 degrees and preferably less than 3 degrees in order to most easily slide through a tight stenosis. By being extraordinarily pushable, the stent delivery system can avoid pre-dilitation of the stenosis and allow direct stenting, thus saving considerable cost and reducing the time for the stenting procedure. The length L1 is approximately 1.0 cm and the length L2 is approximately 1.5 cm. It should be understood however that either of these lengths could be somewhat shorter or longer. Also, the front section of the stent delivery system 10 could be a single cone rather than two conical shapes of different cone angles as shown in FIG. 1.

Although a single elastomer band 13 is shown in FIGS. 1 and 1B, it should be understood that there could also be an elastomer band (not shown) placed just proximal to the proximal end of the stent 15. Either one or both of these elastomer bands would radially expand when the stent 15 is inflated and would shrink back down after the balloon 12 is deflated. A desirable attribute of such an elastomer band would be to assist in folding the balloon 12 when the balloon 12 is deflated. Also, such an elastomer band could be made to include metal particles from a high density metal such as tungsten so as to make the elastomer band radiopaque. By being radiopaque, elastomer bands located at the proximal and distal ends of the stent 15 could assist the interventional cardiologist in the placement of the stent 15.

FIG. 1B illustrates an alternative configuration for the radiopaque marker bands. Specifically, FIG. 1B shows the distal radiopaque marker band 19D2 having its proximal end aligned with the distal end of the stent 15 and the proximal radiopaque marker band 19P2 having its distal end aligned with the proximal end of the stent 15. Since it is more conventional for marker bands to be placed exactly at the proximal and distal edges of the stent 15 as shown in FIG. 1B, the marker bands 19D and 19P of FIG. 1 do not really accomplish that objective However, to increase the diameter of the core wire 14D including the radiopaque marker bands about which the balloon 12 would be wrapped, would increase the outside diameter of the stent 15. To place marker bands exactly at the edges of the stent 15 and not increase the outside diameter of the stent 15, FIG. 1B shows that a groove is placed into the core wire 14D and that groove is filled with approximately 0.001 inch wall thickness of a highly radiopaque metal such as tantalum or gold. This can be accomplished by plating of the radiopaque metal into the grooves shown in FIG. 1B. An alternative to the design of FIG. 1B would be to have a tube of Nitinol for the core wire 14D into which tube cylindrical marker bands would be placed at the appropriate locations. Another means to accomplish radiopaque markers to denote the exact proximal and distal ends of the stent 15 would be to make the elastomer band 16 radiopaque and to add a proximal elastomer band (not shown) that abuts the proximal end of the stent 15 which is also radiopaque. The radiopacity of such elastomer bands could be accomplished by placing a dense metal powder such as tungsten into the elastomer material.

It should also be understood that the distal end of the elastomer band 16 would have a circular shape. However, the proximal end of the elastomer band 16 would have a shape that conforms with essentially zero clearance to the shape of the strut at the proximal end of the stent 15. This design provides a continuous and smooth surface for the outer surface of the stent delivery system 10 so that it can most easily penetrate a tight stenosis. If an elastomer band is also used (though not shown) at the proximal end of the stent 15, then its distal edge would conform to the curvature of the proximal end of the most proximal strut of the stent 15. Another advantage of the elastomer band 16 (and any elastomer band at the proximal end of the balloon 12) is that it would tend to fold the balloon 12 when deflation is required in order to remove the balloon 12 from the deployed stent 15.

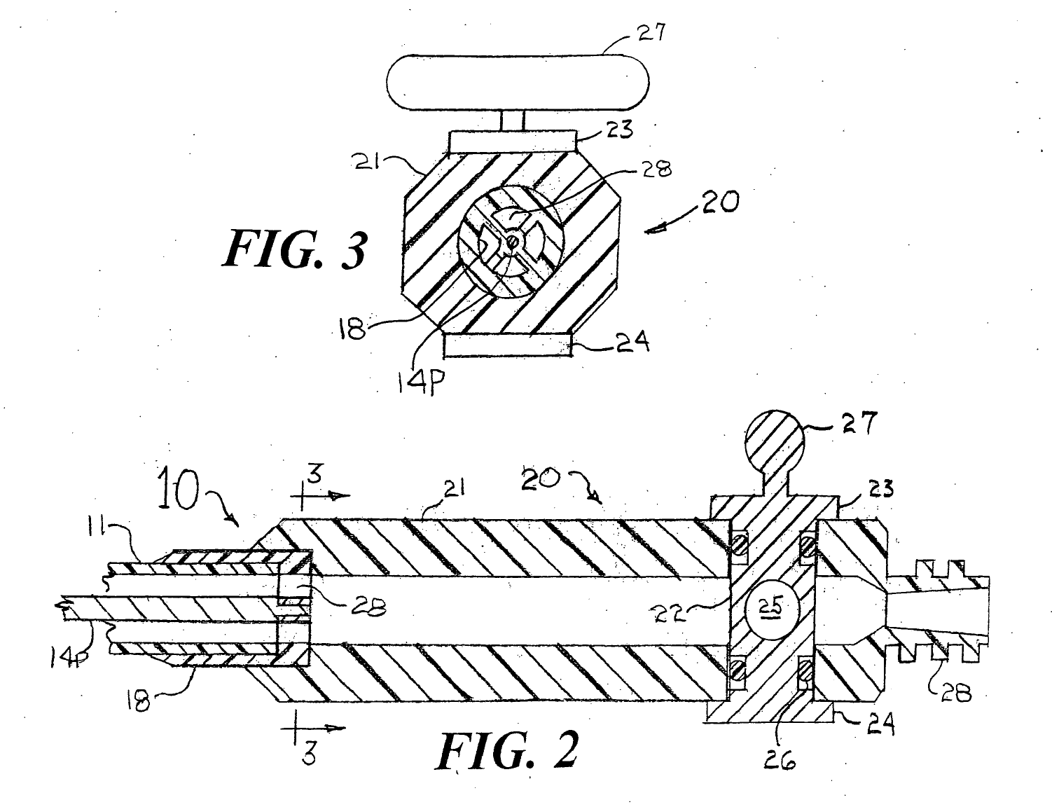

FIG. 2 is a longitudinal cross section of a proximal portion of the stent delivery system 10. The proximal end of the cylindrical tube 11 is fixedly attached within a strain relief 18 that is fixedly attached to the handle body 21 of handle 20 of the stent delivery system 10. A short, smaller diameter section of the proximal wire section 14P is fixedly attached to a central section of the strain relief 18. This construction is shown in FIGS. 2 and 3.

A valve 22 is mounted into the handle body 21 as shown in FIGS. 2 and 3. The valve 22 has an upper shoulder 23 and a lower shoulder 24 that prevent the valve 22 from slipping out of the handle body 21. The valve 22 also has a central passageway 25, “O”-ring seals 26 and a valve handle 27 that is used to rotate the central passageway 25 from the position shown in FIG. 2 to being perpendicular to that position; i.e., to be aligned along the interior lumen of the handle body 21. When the central passageway 25 is as shown in FIG. 2, no liquid can be placed into or out of the stent delivery system 10 and this is called the closed position. When the central passageway 25 is aligned along the lumen of the handle body 21, this is called the open position and a balloon inflation device attached to the Luer fitting 28 can be used to inflate and deflate the balloon 12. A typical procedure would first have the central passageway 25 in the open position and an inflation device (not shown) attached to the Luer fitting 28. The inflation device would then be used to pull a vacuum within the stent delivery system 10. The valve 22 would then be closed, the inflation device removed from the Luer fitting 28 and the interventional cardiologist would advance the stent 15 into the stenosis. The interventional cardiologist would then attach the inflation device containing an inflation fluid (usually saline plus a contrast agent) to the Luer fitting 28, move the central passageway 25 to the open position and inflate the balloon 12 to a pressure of 5 to 25 atmospheres. After the stent 15 is deployed, the inflation device would be used to deflate the balloon 12 and the stent delivery system 10 would be removed from the patient's body.

FIG. 3 is a transverse cross section of the handle body 21 at section 3-3 of FIG. 2. This section shows the distal end of the proximal core wire 14P placed into a central portion of the strain relief 18. Also shown in FIG. 3 is the passageway 28 through which liquid is moved into and out of the stent delivery system 10. The upper shoulder 23, lower shoulder 24 and the valve handle 27 are also shown in FIG. 3. With the valve handle 27 as shown in FIGS. 2 and 3, the valve 22 is in its closed position and when the valve handle 27 is moved to be perpendicular to the position shown in FIGS. 2 and 3, the valve 22 is in the open position. The cross section of the handle body 21 shown in FIG. 3 is octagonal. However, any shape such as a cylindrical or hexagonal which is easily controlled by the interventional cardiologist could be used.

Although this specification and drawings describe a stent delivery system 10 that can be used for placing a stent within a stenosis of a human body, it should also be understood that many of the features of the stent delivery system 10 would be valuable as an angioplasty catheter without a stent. Specifically, the lubricious cone 17 that lies distal to the balloon 12 would aid in placing an angioplasty catheter through a tight stenosis for any use for which angioplasty catheters are now used. Also, the core wire 14 would add to the pushability of an angioplasty catheter used for dilatation of a stenosis.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein

1. A stent delivery system for placing a stent within a vessel of a human body, the system including:
an elongated tube that extends for most of the length of the stent delivery system that is attached at its distal end to the cylindrical proximal portion of an inflatable balloon;
a stent co-axially mounted onto the inflatable balloon;
a front section of the stent delivery system including a small angle cone formed from a polymer material having a lubricious outer surface, the front section being fixedly attached to the cylindrical distal portion of the balloon and also having an outside diameter at the proximal end of the small angle cone that is approximately the same diameter as the outside diameter of the stent mounted onto the inflatable balloon; and
a core wire extending within the inflatable balloon and also extending within the front section of the stent delivery system.

2. The system of claim 1 where the tube is made from hypo tubing or from a hollow stranded core.

3. The system of claim 1 where the tube is made from a polymer material with metal reinforcing.

4. The system of claim 1 where the stent is made from a metal selected from the group consisting of stainless steel, cobalt-chromium alloy and tantalum.

5. The system of claim 1 where the stent is a drug eluting stent.

6. The system of claim 1 where the stent is coated with heparin.

7. The system of claim 1 where the stent is a bioabsorbable stent.

8. The system of claim 1 where the small angle cone of the front section of the stent delivery system is made from a polymer material selected from the group consisting of polyurethane, silicone rubber, polyethylene, PeBax, polyamide, PTFE or Nylon.

9. The system of claim 1 where the small angle cone of the front section of the stent delivery system has a hydrophilic coating to enhance its lubricity.

10. The system of claim 1 where the cone apex angle of the small angle cone is less than 10 degrees.

11. The system of claim 1 where the cone apex angle of the small angle cone is less than 3 degrees.

12. The system of claim 1 where an elastomer band is placed onto the balloon, the elastomer band being situated between the proximal end of the small angle cone and the distal end of the stent.

13. The system of claim 12 where the elastomer band includes radiopaque metal particles that make the elastomer band readily viewable by fluoroscopy.

14. The system of claim 12 where the proximal edge of the elastomer band is shaped to conform to the shape of the distal edge of the stent.

15. The system of claim 12 including an elastomer band that is placed just proximal to the proximal end of the stent.

16. The system of claim 15 where both elastomer bands include radiopaque metal particles that make each elastomer band readily viewable by fluoroscopy.

17. The system of claim 1 where the core wire is formed from a shape memory alloy.

18. The system of claim 17 where the shape memory alloy is Nitinol.

19. The system of claim 1 where the core wire within the front section of the stent delivery system is a shape memory alloy that has a transition temperature that is higher than body temperature.

20. The system of claim 1 where the core wire that extends within the inflatable balloon is formed from a shape memory alloy that has a transition temperature that is lower than body temperature.

21. The system of claim 1 where the core wire extends for nearly the entire length of the stent delivery system, the core wire having a smaller outside diameter within the front section and the balloon and a larger outside diameter where it extends in a proximal direction beyond the proximal end of the balloon.

22. The system of claim 1 further including a handle located at a proximal portion of the stent delivery system, the handle being used by an interventional cardiologist to guide the stent delivery system through the vasculature of a human body.

23. The system of claim 22 where the core wire is attached at its proximal end to the handle that is used by an interventional cardiologist to guide the stent delivery system through the vasculature of a human body.

24. The system of claim 22 where the handle includes a valve that can be placed in the open position to allow liquid into or out of the stent delivery system or placed in the closed position to prevent liquid from going into or out of the stent delivery system.

25. An angioplasty catheter for dilating a stenosis of a vessel of a human subject, the angioplasty catheter including:
an elongated tube extending for most of the length of the angioplasty catheter, the elongated tube having a distal end onto which is mounted an inflatable balloon;
a front section that is situated distal to the distal end of the balloon, the front section having a small apex angle polymer cone, the cone having an outside diameter at its proximal end that is approximately equal to the outside diameter of the folded balloon, and
a core wire that extends axially through the front section of the angioplasty catheter and also extends through the entire length of the inflatable balloon.
